(19) United States
(12) Patent Application Publication
(10) Pub. No.: US 2008/0286351 A1
(43) Pub. Date: Nov. 20, 2008

(54) Pegylated Cardiolipin Analogs, Methods of Synthesis, and Uses Thereof

(76) Inventors: Moghis U. Ahmad, Wadsworth, IL (US); Murali K. Ukkalam, Blacksburg, VA (US); Shoukath M. Ali, Lake Bluff, IL (US); Imran Ahmad, Wadsworth, IL (US)

Correspondence Address: NEOPHARM, INC., 101 WAUKEGAN ROAD, SUITE 970, LAKE BLUFF, IL, 60044, US

(21) Appl. No.: 11/631,468

(22) PCT Filed: Jun. 29, 2005
(86) PCT No.: PCT/US05/23286

§ 371 Date: May 14, 2008

Related U.S. Application Data
(60) Provisional application No. 60/583,833, filed on Jun. 29, 2004.

(51) Int. Cl. A61K 9/127; C07C 69/003; A61K 31/661; A61P 35/00

(52) U.S. Cl. 424/450; 560/129; 514/121

(57) ABSTRACT

The invention provides synthetic methods for PEGylated cardiolipins with varying linkers. The methods can be employed to prepare PEGylated cardiolipin with different fatty acid and/or alkyl chain length with or without unsaturation. The PEGylated cardiolipin, prepared by the present methods, can be incorporated into liposomes that can also include active agents such as hydrophilic or hydrophobic drugs for the treatment of human and animal diseases. In addition, the PEGylated cardiolipin can be incorporated into liposomes that include compounds for therapeutic and diagnostic imaging. The use of such liposomes with PEGylated cardiolipin prolongs the period of liposomal circulation without disrupting the lipid bilayer.

### CROSS-REFERENCE TO RELATED APPLICATIONS

This patent application claims the benefit of U.S. Provisional Patent Application No. 60/583,833, filed on Jun. 29, 2004, the disclosure of which is incorporated herein in its entirety.

### FIELD OF THE INVENTION

This invention relates to novel PEGylated cardiolipin analogues and variants, the methods for preparing them, and to liposome compositions that contain them. The invention also relates to liposome formulations containing therapeutic agents and their use in drug delivery for the treatment of mammalian diseases.

### BACKGROUND OF THE INVENTION

Liposome formulations have the capacity to increase the solubility of hydrophobic drugs in aqueous solutions. They often reduce the side effects associated with drug therapy and provide flexible tools for developing new formulations of active agents.

Liposomes have been proposed as a drug carrier for intravenously (IV) administered compounds, including both imaging and therapeutic compounds (see Harrington et al., Clinical Cancer Research, 7, 243-254, (2001)). However, the use of liposomes for site-specific targeting via bloodstream has been severely restricted by the rapid clearance of liposomes by cells of the reticuloendothelial system (RES). Typically, the RES will remove 80-95% of a dose of IV injected liposomes within one hour, effectively out-competing the selected target site for uptake of the liposomes (U.S. Patent Application 2003/0113369; Harrington et al., Clinical Cancer Research, 6, 2528-2537, (2000)).

Methods that have been employed to improve the stability of circulating liposomes include incorporation of glycolipids or cholesterol within lipid compositions of liposomes. The disadvantages of using cholesterol or other high phase transition lipids include the decrease in the permeability of the vesicles membrane to water which results in a decreased relaxivity for the entrapment of active agents (U.S. Pat. No. 6,132,763).

The presence of PEGylated lipids in the bilayer membrane of sterically stabilized liposomes effectively furnishes a steric barrier against interactions with plasma proteins and cell surface receptors that are responsible for the rapid intravascular destabilization/rupture and RES clearance seen after IV administration of conventional liposomes (see Harrington et al., Clinical Cancer Research, 5, 243-254, (2001)). As a result, the use of PEGylated liposomes have shown a prolonged circulation half-life and have resulted in effective tumor targeting (see Huang et al., Cancer Res. 52, 5135-5145, (1992); Huang et al., Am. J. Pathol 143, 10-14 (1993)); and therapeutic efficacy (see Harrington et al., Clin. Oncol. (R. Coll. Radiol) 12, 2-15, (2000)) in a number of animal models. The superiority of a polyethylene glycol (PEG)-derivatized phospholipid delivery system (see Working et al., J. Pharmacol. Exp. Ther., 289, 1128-1133 (1999); Gabizon et al., J Controlled Release, 53, 275-279 (1999)) at low doses is well documented.

It has been shown that the covalent linkage of the PEG group to the external surface of liposomes can extend the circulation life-time of the liposomes without disrupting the lipid bilayer. In order to prepare these PEGylated liposomes, liposomes have been treated with a reactive derivative of polyethylene glycol in aqueous solution at ambient or physiological conditions (U.S. Pat. No. 6,132,763).

PEG-derivatized liposomes are known to evade the cells of the mononuclear phagocyte system (MPS) and are also as called “Stealth liposomes” (see Woodle et al., Biochem. Biophys. Acta, 1113, 171 (1992); Lasic et al., Chem. Rev., 95, 2601 (1995)). Stealth liposomes are prepared by the covalent attachment of a hydrophilic polymer, such as a PEG group, to the liposome surface.

Considering the state of the art, there is a need for an improved method of PEGylating liposomes and for reagents that can be used to generate liposomes.

### BRIEF SUMMARY OF THE INVENTION

The invention provides PEGylated cardiolipin and methods for the synthesis of PEGylated cardiolipin using a PEG group of molecular weight ranging from 200 to 50,000 daltons. The invention also provides different methods for the preparation of PEGylated cardiolipin having varying saturated or unsaturated fatty acid or alkyl chain lengths. The PEGylated cardiolipin can conveniently be incorporated into liposomes that can also include active agents such as hydrophobic and hydrophilic drugs. Such PEGylated liposomes can be used for drug delivery for the treatment of human and animal diseases.

Cardiolipin (also known as diphosphatidyl glycerol), constitutes a class of complex anionic phospholipids that is typically purified from the cell membranes of tissues associated with high metabolic activity, including the mitochondria of heart and skeletal muscles. Cardiolipin and cardiolipin analogues can be obtained by chemical synthesis (PCT/US03/16412; PCT/US03/27806; Krishna et al., Tetrahedron Lett. 45, 2077-2079 (2004)), and cardiolipin and cardiolipin analogues have been employed in liposomal preparations. However, the synthesis of PEG-derivatized cardiolipin analogues and their usage in liposome formulations have not been reported so far. The addition of PEGylated cardiolipin analogues may increase the circulation lifetime of liposomes without disrupting the lipid bilayer. Advantages of the present invention, as well as additional inventive features, will be apparent from the description of the invention provided herein.

### BRIEF DESCRIPTION OF THE DRAWINGS

FIG. 1 depicts a synthetic scheme for PEGylated cardiolipin. In the figure, “Bn” indicates a benzyl group.

FIG. 2 depicts a synthetic scheme for PEGylated cardiolipin.

FIG. 3 depicts a synthetic scheme for PEGylated cardiolipin.

FIG. 4 depicts a synthetic scheme for PEGylated cardiolipin.

### DETAILED DESCRIPTION OF THE INVENTION

The present invention provides a PEGylated cardiolipin molecule of the general formula I.

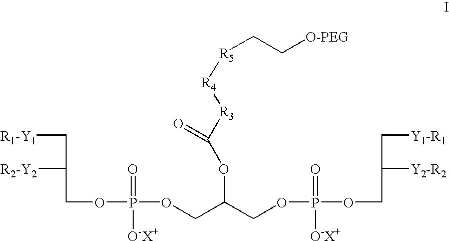

Wherein Y₁ and Y₂ are the same or different and are —O—C(O)—, —O—, —S—, —NH—C(O)— or the like;
R₁ and R₂ are the same or different and are H, saturated and/or unsaturated alkyl group;
R₃, R₄, R₅ are the same or different and are —O—, —C(O)—, —NR— wherein R is H or an alkyl group ranging from C₁-C₁₀, (CH₂), where n=0-10 or substituted (CH₂)ₙ where n=0-10;
X is hydrogen, ammonium, sodium, potassium, calcium, barium ion or any non-toxic cation; and the PEG (polyethylene glycol) group is a long chain, linear or branched synthetic polymer with a molecular weight ranging from 200-50,000 daltons.

The present invention also provides a PEGylated cardiolipin molecule of the general formula II.

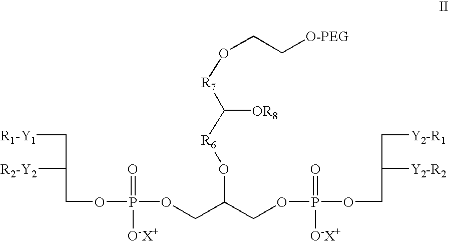

Wherein Y₁ and Y₂ are the same or different and are —O—C(O)—, —O—, —S—, —NH—C(O)— or the like;
R₁ and R₂ are the same or different and are H, saturated and/or unsaturated alkyl group;
R₆ and R₇ are the same or different and are (CH₂)ₙ where n=1-10 or substituted (CH₂)ₙ where n=1-10.
R₈ is H, alkyl, substituted alkyl, cycloalkyl, substituted cycloalkyl, alkyloxy or polyalkyloxy group;
X is hydrogen, ammonium, sodium, potassium, calcium, barium ion or any non-toxic cation and the PEG (polyethylene glycol) group is a long chain, linear or branched synthetic polymer with a molecular weight ranging from 200-50,000 daltons.

In Formulas I and II, at least one of R₁ or R₂ is preferably a saturated or unsaturated alkyl group having between 1 and 34 carbon atoms. In a preferred embodiment, in Formulas I and II, R₁ and R₂ are the same and include from C₁ to C₃₄ saturated and/or unsaturated alkyl group, preferably between 6 and 24 carbon atoms and more preferably between 12 and 24 carbon atoms. The terms “alkyl” encompasses saturated or unsaturated straight chain and branched-chain hydrocarbon moieties. Unless otherwise specified herein, saturated and unsaturated alkyl moieties can have any suitable number of carbon atoms, e.g. C₁-C₅₀, more typically C₄-C₃₄, such as C₆-C₂₄ or C₆-C₁₂ or even C₁₂-C₂₄. Substituted, saturated or unsaturated cycloalkyl groups can have any suitable number of carbon atoms but are typically C₃-C₈, such as C₄-C₆. The term “substituted alkyl” comprises alkyl groups further bearing one or more substituents selected from hydroxyl, alkoxy (of a lower alkyl group), mercapto (of a lower alkyl group), cycloalkyl, substituted cycloalkyl, halogen, cyano, nitro, amino, amido, imino, thio, —C(O)H, acyl, oxyacyl (of a lower acyl) carboxyl and the like. X is more preferably a hydrogen or ammonium ion.

In another preferred embodiment, in Formula I, R₃, R₄, R₅ are oxygen, nitrogen optionally substituted with H, or alkyl chain of length 0-10, carbonyl group, methylene groups (CH₂)ₙ optionally substituted with alkyl group, halogen, cyano, nitro, amino, hydroxyl and the like where n=1-10.

In another preferred embodiment, in Formula II, R₆ and R₇ are same or different and are methylene groups (CH₂)ₙ optionally substituted with alkyl group, halogen, cyano, amino and the like where n=1-10.

In another preferred embodiment, in Formula II, R₈ is H, alkyl, substituted alkyl, cycloalkyl, substituted cycloalkyl, alkyloxy, polyalkyloxy such as PEGylated ether containing 1 to about 500 alkyloxy mers.

In another preferred embodiment, the PEG (polyethylene glycol) group in Formulas I and II is a long chain, linear or branched synthetic polymer composed of ethylene oxide units, HO(CH₂CH₂O)ₙCH₂CH₂OCH₃, in which n is typically between about 1 and about 1000 (such as between about 1 and about 500) or otherwise can vary to provide compounds with molecular weights (M.W.) from 200-50,000 daltons.

In a preferred embodiment for the compound of general Formula I is a PEGylated cardiolipin of structure III.

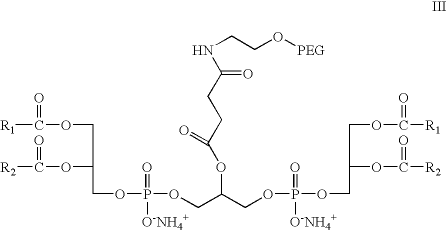

Wherein R₁ and R₂ are the same or different and are H, C₁ to C₃₄ saturated and/or unsaturated alkyl groups.

Another preferred embodiment for the compound of general Formula I is a PEGylated cardiolipin of structure IV.

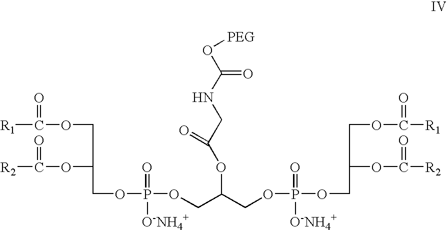

Wherein R₁ and R₂ are the same or different and are H, C₁ to C₃₄ saturated and/or unsaturated alkyl groups.

Another preferred embodiment for the compound of general Formula I is a PEGylated cardiolipin of structure V.

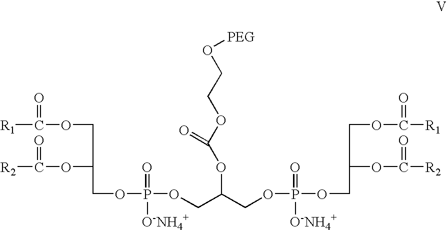

Wherein R₁ and R₂ are the same or different and are H, C₁ to C₃₄ saturated and/or unsaturated alkyl groups.

Another preferred embodiment for the compound of general Formula I is a PEGylated cardiolipin of structure VI.

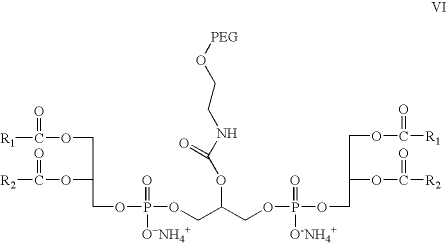

Wherein R₁ and R₂ are the same or different and are H, C₁ to C₃₄ saturated and/or unsaturated alkyl groups.

A preferred embodiment for the compound of general Formula II is a PEGylated cardiolipin of structure VII.

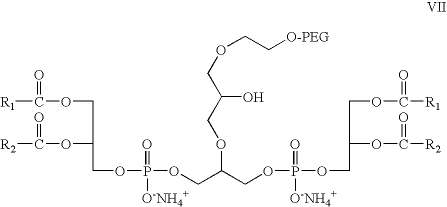

Wherein R₁ and R₂ are the same or different and are H, C₁ to C₃₄ saturated and/or unsaturated alkyl groups.

PEGylated cardiolipin analogues, such as those of Formulas I and II, can be prepared by any desired method, and the invention provides methods of preparing PEGylated cardiolipin and analogues thereof. The inventive method comprises reacting a functional group of cardiolipin, or a functional group of a linker with a PEG-reagent. The reactive functional group of the cardiolipin and the linker includes any suitable functional groups that will react with a polyethylene glycol reagent (PEG reagent). The PEG-reagent comprises a linear or branched polyether terminated with a reactive functional group such as a hydroxyl, an amino group, a carboxyl group, an isocyanate group, a carbonate group, and the like. The PEG-reagents can also have an activating group such as dichlorotriazine, tresylate, benzotriazole, carbamate, carbonyl imidazole, succinimidyl succinate, N-hydroxysuccinimide, succinimidyl glutarate or p-nitrophenyl carbamate. Preferred PEG-reagents are PEG N-hydroxysuccinimide (PEG-NHS), PEG Succinimidyl Glutarate (PEG-SG), PEGylated p-nitrophenyl carbonate (PEG-NPC—formula 14) PEGylated isocynate (PEG-NC—formula 15) and PEGylated epoxide (formula 16).

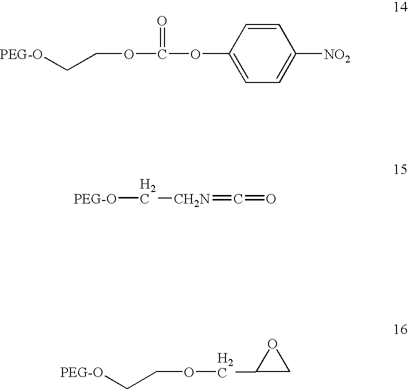

In a preferred embodiment, the inventive method involves linking a PEG group to the central glycerol unit of a cardiolipin molecule or cardiolipin molecule analogue. For example, a linker can be attached to the cardiolipin molecule (or an analogue thereof) by reacting the central hydroxyl group of a cardiolipin precursor of formula 1 with a cyclic anhydride in an inert solvent in the presence of a base.

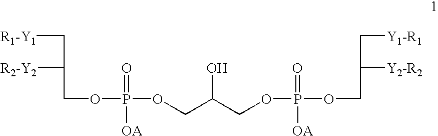

Wherein A is a protecting group preferably a benzyl or methyl group;
R₁ and R₂ can be any suitable alkyl group, are the same or different, can be saturated and/or unsaturated, and preferably have 1 to 34 carbon atoms;
the linker comprises an alkyl, aralkyl, aryl substituted alkyl, substituted aralkyl, or substituted aryl where the substituents are preferably CHO, COOH, OH, NHR, SH, CONHR, C(O)OR, NCO, NO₂, tosylate, mesylate or halogens.

In this context, alkyl groups are preferably C₁-C₁₀, aralkyl groups preferably comprise 1 to 3 aromatic rings, and more preferably one aromatic ring, wherein each ring preferably has 5 to 8 ring atoms including 0 to 3 heteroatoms, and more preferably wherein each ring has 5 to 6 ring atoms. Examples of suitable cyclic anhydrides include succinic anhydride and glutaric anhydrides, but other cyclic anhydrides also can be suitably employed. The resulting cardiolipin precursor, containing a linker, is then reacted with the PEG-reagent, having a reactive functional group. The reaction between the cardiolipin precursor containing the linker and the PEG-reagent can occur in the presence or the absence of an activating group. In this embodiment, preferred PEG-reagents have hydroxyl, amino, carboxyl, isocyanate, carbonate functional groups. Especially preferred PEG-reagents for use in this embodiment of the inventive method include PEG-NHS and PEG-SG. Following the reaction between the cardiolipin precursor containing the linker and the PEG-reagent, the protecting groups are removed.

In another preferred embodiment, the central hydroxyl group of a cardiolipin precursor of formula 1 is reacted with a PEG-reagent. In this embodiment, a preferred PEG-reagent is PEG-NPC (formula 14), in which instance, the reaction preferably is conducted at an alkaline pH (most preferably at a pH of between about 8 and about 9). Other preferred PEG-reagents for use in this method include PEG-NC and PEGylated epoxide of formulas 15 and 16. Following the reaction between the cardiolipin precursor containing the linker and the PEG-reagent, the protecting groups are removed.

In another preferred embodiment, a phosphoramidite derivative of 1,2-substituted glycerol of formula 7 is treated with 2-substituted glycerol of formula 8 in the presence of an agent such as 1-H-tetrazole, 4,5-dicyanoimidazole, or the like.

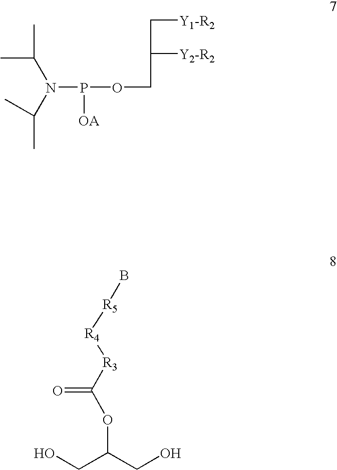

Following this reaction, the protecting group B is removed (such as with an acid in an inert solvent), which results in a free amino group. The free amino group is reacted with a PEG-reagent in the presence of N,N-dimethylamino pyridine (DMAP) in an inert solvent. In this embodiment, preferred PEG-reagents have hydroxyl, amino, carboxyl, isocyanate, carbonate functional groups. Especially preferred PEG-reagents for use in this embodiment of the inventive method include PEG-NHS and PEG-SG.

In another preferred embodiment, a phosphoramidite derivative of 1,2-substituted glycerol of formula 10 is treated with 2-substituted glycerol of formula 8 in the presence of pyridinium perbromide in an inert solvent or the like and containing a suitable base.

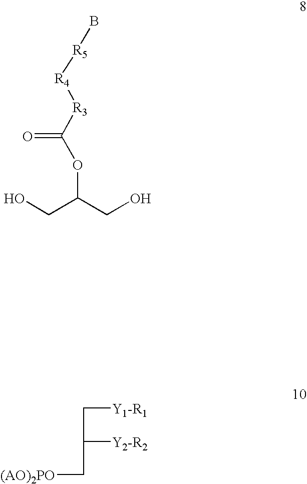

Thereafter, the protecting group B is removed (such as with an acid in an inert solvent), which results in a free amino group. The free amino group is reacted with a PEG-reagent in the presence of DMAP in an inert solvent. In this embodiment, preferred PEG-reagents have hydroxyl, amino, carboxyl, isocyanate, and/or carbonate functional groups. Especially preferred PEG-reagents for use in this embodiment of the inventive method include PEG-NHS and PEG-SG.

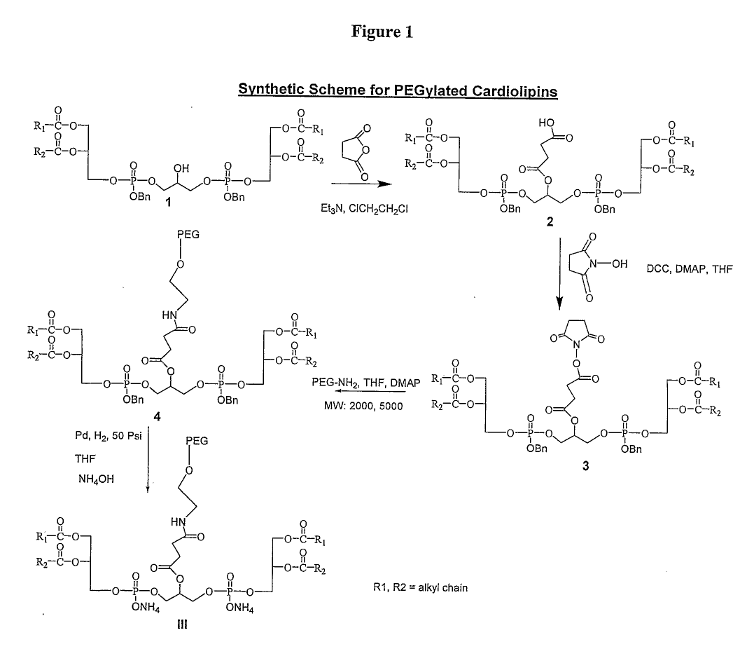

One preferred method of the present invention is set forth in FIG. 1, which depicts an approach to the synthesis of PEGylated cardiolipin. In this method, cardiolipin derivative 1 is synthesized using a phosphate or phosphoramidite method (see PCT/US03/16412; PCT/US03/27806). This intermediate is reacted with succinic anhydride in an inert solvent (for example, 1,2-dichloroethane and the like) in presence of base (for example, triethyl amine and the like) to provide PEGylated cardiolipin precursor 2.

The carboxyl group of PEGylated cardiolipin precursor 2 can be activated with any suitable activating agent. For example, N-hydroxy-succinimide can be used to activate the carboxyl group in presence of N,N-dicyclohexyl carbodimide (DCC) and N,N-dimethylamino pyridine (DMAP) in THF to yield the precursor in activated form. Coupling of the activated precursor 3 with PEGylated amine in presence of DMAP in tetrahydrofuran (THF) provides 4.

Removal of benzyl groups to yield PEGylated cardiolipin III can be accomplished by any suitable reagents. For example, the benzyl group can be removed by hydrogenation in presence of palladium catalyst.

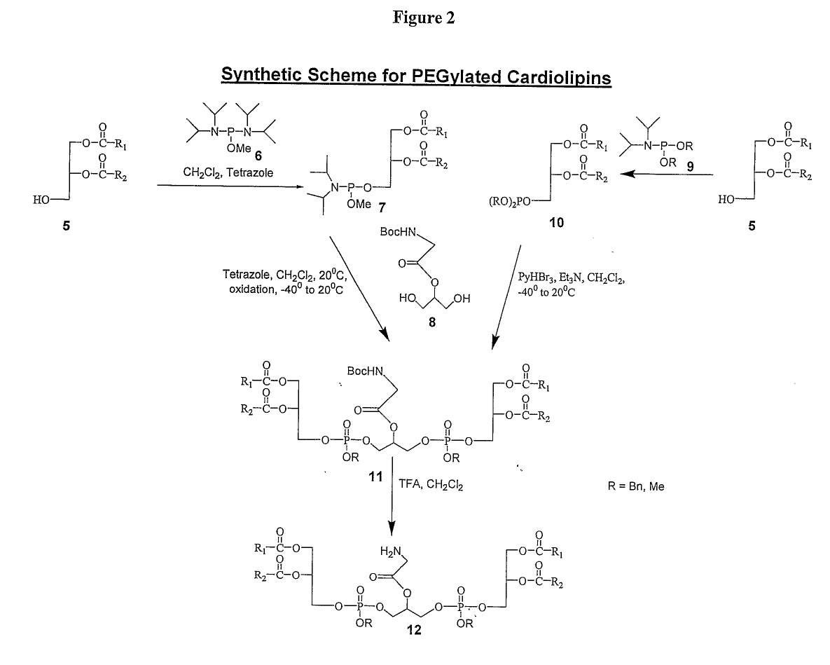

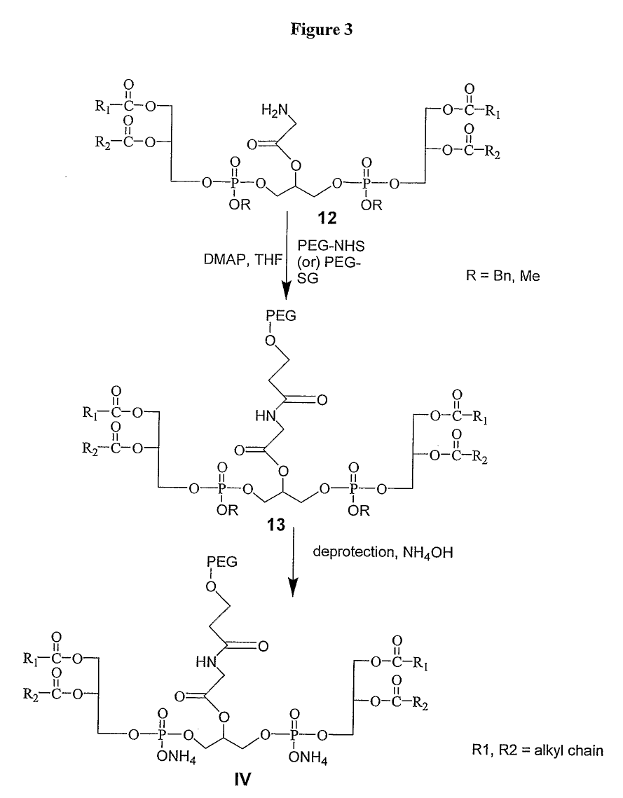

Another embodiment of the present invention is represented in FIGS. 2-3. In this method, any suitable phosphoramidite reagent, such as N,N,N,N-tetraisopropylaminophosphorodimidite 6 is reacted with 1,2-O-diacyl glycerol 5 in presence of tetrazole in an inert solvent (for example, dichloromethane and the like) to provide intermediate 7 which subsequently reacted with 2-substituted glycerol (for example, 2-[(N-ter-butoxycarbonyl)aminoacetyl]glycerol) 8 to give Intermediate 11.

Alternatively, 1,2-di-O-acyl glycerol 5 can be phosphorylated using phosphoramidite reagent 9 to yield phosphate triesters 10, which can be coupled with 2-substituted glycerol such as 2-[(N-ter-butoxycarbonyl)aminoacetyl]glycerol 8 in the presence of pyridinium perbromide in an inert solvent such as dichloromethane and the like, containing a suitable base such as triethylamine and the like, to provide intermediate 11. The preferred coupling reagent in this context of synthetic methods is dibenzyldiisopropylphosphoramidite.

Removal of the ter-butoxycarbonyl group from intermediate 11 can be accomplished by any suitable acid such as trifluoroacetic acid and the like, in any inert solvent such as dichloromethane and the like, to give Intermediate 12.

Reaction of the free amino group of intermediate 12 with PEG-NHS or PEG-SG in presence of N,N-dimethylamino pyridine (DMAP) in an inert solvent such as THF and the like, provides PEGylated cardiolipin precursor 13 (FIG. 3).

The deprotection can be achieved by any suitable method depending on the protecting group. For example a benzyl group can be removed by catalytic hydrogenolysis or by treatment with sodium iodide; cyanoethyl and fluorenylmethyl groups by treatment with a tertiary base such as triethylamine; a silyl group can be deprotected with fluoride ion or acidic medium.

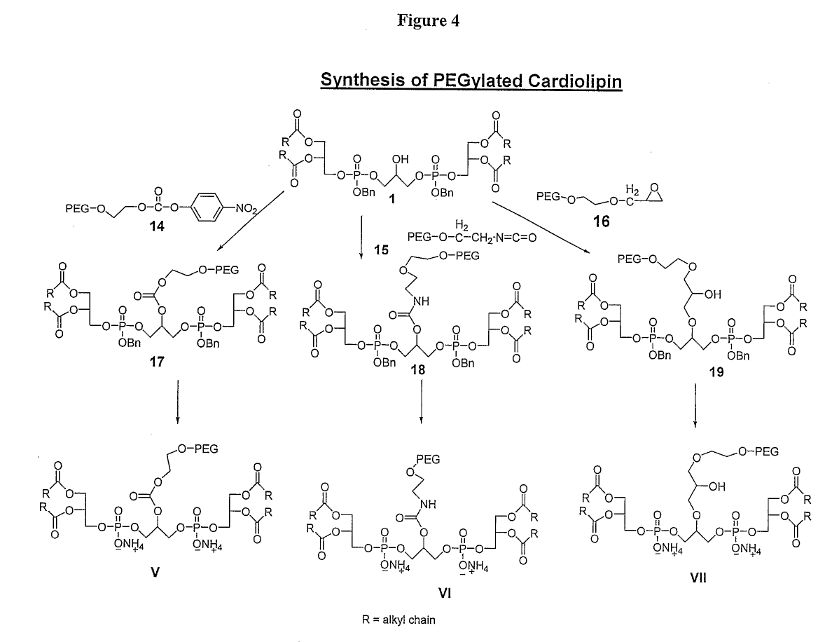

Another embodiment of the present invention, represented in FIG. 4, involves reaction of cardiolipin intermediate 1 with PEGylated p-nitrophenyl carbonate (PEG-NPC) 14, PEGylated isocyanate (PEG-NC) 15, and PEGylated epoxide 16 at pH 8-9, to give 17, 18, and 19 respectively. Removal of benzyl groups by any desired methods provides PEGylated cardiolipins V, VI, and VII respectively.

The synthetic methods described herein can be modified in any suitable manner. For example, the carboxyl group can be activated by variety of activating agents, including 1,1′-carbonyldimidazole; 1-ethyl-3-[3-(dimethylamino)propyl]-carbodimide (EDCI); hydroxybenzotriazole (HOBT); methyl chloroformate and the like. Similarly, amine protection is not limited to ter-butoxycarbonyl group (BOC) but also includes benzyloxycarbonyl (Cbz), trityl, pthalimide and the like.

The PEGylating group for use in the inventive method can be any PEG derivative, which is capable of reacting with hydroxyl or amino group of cardiolipin or functional group of any linker.

The solvent for PEGylation reaction in the inventive method includes any solvent preferably a polar aprotic solvent such as N,N-dimethylformamide (DMF), dimethylsulfoxide (DMSO), pyridine, tetrahydrofuran (THF), dichloromethane, chloroform, 1,2-dichloroethane, dioxane and the like.

The described methods can be used to prepare a variety of novel PEGylated cardiolipin species. For example, the methods can be used to prepare PEGylated cardiolipin in pure form containing any fatty acid chain. Preferred fatty acids range from carbon chain lengths of about C₂ to C₃₄, preferably between about C₄ and about C₂₄, and include tetranoic acid (C_{4:0}), pentanoic acid (C_{5:0}), hexanoic acid (C_{6:0}), heptanoic acid (C_{7:0}), octanoic acid (C_{8:0}), nonanoic acid (C_{9:0}), decanoic acid (C_{10:0}), undecanoic acid (C_{11:0}), dodecanoic acid (C_{12:0}), tridecanoic acid (C_{13:0}), tetradecanoic (myristic) acid (C_{14:0}), pentadecanoic acid (C_{15:0}), hexadecanoic (palmatic) acid (C_{16:0}), heptadecanoic acid (C_{17:0}), octadecanoic (stearic) acid (C_{18:0}), nonadecanoic acid (C_{19:0}), eicosanoic (arachidic) acid (C_{20:0}), heneicosanoic acid (C_{21:0}), docosanoic (behenic) acid (C_{22:0}), tricosanoic acid (C_{23:0}), tetracosanoic acid (C_{24:0}), 10-undecenoic acid (C_{11:1}), 11-dodecenoic acid (C_{12:1}), 12-tridecenoic acid (C_{13:1}), myristoleic acid (C_{14:1}), 10-pentadecenoic acid (C_{15:1}), palmitoleic acid (C_{16:1}), oleic acid (C_{18:1}), linoleic acid (C_{18:2}), linolenic acid (C_{18:3}), eicosenoic acid (C_{20:1}), eicosdienoic acid (C_{20:2}), eicosatrienoic acid (C_{20:3}), arachidonic acid (cis-5,8,11,14-eicosatetraenoic acid), and cis-5,8,11,14,17-eicosapentaenoic acid, among others. For ether analogs, the alkyl chain will also range from C₂ to C₃₄ preferably between about C₄ and about C₂₄. Other fatty acid chains also can be employed as R₁ and/or R₂ substituents. Examples of such include saturated fatty acids such as ethanoic (or acetic) acid, propanoic (or propionic) acid, butanoic (or butyric) acid, hexacosanoic (or cerotic) acid, octacosanoic (or montanic) acid, triacontanoic (or melissic) acid, dotriacontanoic (or lacceroic) acid, tetratriacontanoic (or gheddic) acid, pentatriacontanoic (or ceroplastic) acid, and the like; monoethenoic unsaturated fatty acids such as trans-2-butenoic (or crotonic) acid, cis-2-butenoic (or isocrotonoic) acid, 2-hexenoic (or isohydrosorbic) acid, 4-decanoic (or obtusilic) acid, 9-decanoic (or caproleic) acid, 4-dodecenoic (or linderic) acid, 5-dodecenoic (or denticetic) acid, 9-dodecenoic (or lauroleic) acid, 4-tetradecenoic (or tsuzuic) acid, 5-tetradecenoic (or physeteric) acid, 6-octadecenoic (or petroselenic) acid, trans-9-octadecenoic (or elaidic) acid, trans-1-octadecenoic (or vaccinic) acid, 9-eicosenoic (or gadoleic) acid, 11-eicosenoic (or gondoic) acid, 11-docosenoic (or cetoleic) acid, 13-decosenoic (or erucic) acid, 15-tetracosenoic (or nervonic) acid, 17-hexacosenoic (or ximenic) acid, 21-triacontenoic (or lumequeic) acid, and the like; dienoic unsaturated fatty acids such as 2,4-pentadienoic (or β-vinylacrylic) acid, 2,4-hexadienoic (or sorbic) acid, 2,4-decadienoic (or stillingic) acid, 2,4-dodecadienoic acid, 9,12-hexadecadienoic acid, cis-9, cis-12-octadecadienoic (or α-linoleic) acid, trans-9, trans-12-octadecadienoic (or linlolelaidic) acid, trans-10, trans-12-octadecadienoic acid, 11,14-eicosadienoic acid, 13,16-docosadienoic acid, 17,20-hexacosadienoic acid and the like; trienoic unsaturated fatty acids such as 6,10,14-hexadecatrienoic (or hiragonic) acid, 7,10,13-hexadecatrienoic acid, cis-6, cis-9-cis-12-octadecatrienoic (or γ-linoleic) acid, trans-8, trans-10-trans-12-octadecatrienoic (or β-calendic) acid, cis-8, trans-10-cis-12-octadecatrienoic acid, cis-9, cis-12-cis-15-octadecatrienoic (or α-linolenic) acid, trans-9, trans-12-trans-15-octadecatrienoic (or α-linolenelaidic) acid, cis-9, trans-10-trans-13-octadecatrienoic (or α-eleostearic) acid, trans-9, trans-11-trans-13-octadecatrienoic (or β-eleostearic) acid, cis-9, trans-1-cis-13-octadecatrienoic (or punicic) acid, 5,8,11-eicosatrienoic acid, 8,11,14-eicosatrienoic acid and the like; tetraenoic unsaturated fatty acids such as 4,8,11,14-hexadecatetraenoic acid, 6,9,12,15-hexadecatetraenoic acid, 4,8,12,15-octadecatetraenoic (or moroctic) acid, 6,9,12,15-octadecatetraenoic acid, 9,11,13,15-octadecatetraenoic (or α- or β-parinaric) acid, 9,12,15,18-octadecatetraenoic acid, 4,8,12,16-eicosatetraenoic acid, 6,10,14,18-eicosatetraenoic acid, 4,7,10,13-docasatetraenoic acid, 7,10,13,16-docosatetraenoic acid, 8,12,16,19-docosatetraenoic acid and the like; penta- and hexa-enoic unsaturated fatty acids such as 4,8,12,15,18-eicosapentaenoic (or timnodonic) acid, 4,7,10,13,16-docosapentaenoic acid, 4,8,12,15,19-docosapentaenoic (or clupanodonic) acid, 7,10,13,16,19-docosapentaenoic, 4,7,10,13,16,19-docosahexaenoic acid, 4,8,12,15,18,21-tetracosahexaenoic (or nisinic) acid and the like; branched-chain fatty acids such as 3-methylbutanoic (or isovaleric) acid, 8-methyldodecanoic acid, 10-methylundecanoic (or isolauric) acid, 11-methyldodecanoic (or isoundecylic) acid, 12-methyltridecanoic (or isomyristic) acid, 13-methyltetradecanoic (or isopentadecylic) acid, 14-methylpentadecanoic (or isopalmitic) acid, 15-methylhexadecanoic, 10-methylheptadecanoic acid, 16-methylheptadecanoic (or isostearic) acid, 18-methylnonadecanoic (or isoarachidic) acid, 20-methylheneicosanoic (or isobehenic) acid, 22-methyltricosanoic (or isolignoceric) acid, 24-methylpentacosanoic (or isocerotic) acid, 26-methylheptacosanoic (or isomonatonic) acid, 2,4,6-trimethyloctacosanoic (or mycoceranic or mycoserosic) acid, 2-methyl-cis-2-butenoic(angelic) acid, 2-methyl-trans-2-butenoic (or tiglic) acid, 4-methyl-3-pentenoic (or pyroterebic) acid and the like. For ether analogs, the alkyl chain also will range from carbon chain lengths of C₄ to C₃₄.

One preferred use for the inventive PEGylated cardiolipin is in the preparation of liposomes and other lipid-containing formulations. Accordingly, the invention provides a method of preparing a liposome comprising preparing a PEGylated cardiolipin (such as via methods described herein), and including the PEGylated cardiolipin in a liposome. Liposomes according to the present invention can be prepared by any suitable technique, such as those known to those of ordinary skill in the art. For example, lipophilic liposome-forming ingredients, such as phosphatidylcholine, a PEGylated cardiolipin or analog prepared by the methods described above, cholesterol and α-tocopherol can be dissolved or dispersed in a suitable solvent or combination of solvents and dried. The amount of PEGylated cardiolipin in liposome can be controlled by varying the composition of lipid and or other components in it.

Other preferred lipophilic agents include a phosphatidylcholine, a sterol and a tocopherol, and, in addition to the PEGylated cardiolipin (or analogue thereof), the liposomes preferably include such lipophilic agents. Other preferred lipophilic agents include one or more phospholipids, such as tetramyristoyl cardiolipin, dioleoylphosphatidylcholine (DOPC), dipalmytoylphosphatidylcholine (DPPC), disteroylphosphatidylcholine (DSPC), dimyristoylphosphatidylcholine (DMPC), phosphatidylcholine, dioleoylphosphatidylethanolamine (DOPE) and mixtures thereof. Other preferred lipophilic agents include one or more phosphatidylglycerols, such as dimyristoylphosphatidylglycerol, distearoylphosphatidylglycerol, dioleoylphoshatidylglycerol, dipalmitoylphosphatidylglycerol, diarachidonoylphosphatidylglycerol, and mixtures thereof. Other preferred lipophilic agents include one or more sterols, such as cholesterol, coprostanol, cholestanol, cholestane, cholesterol hemisuccinate, cholesterol sulfate, and mixtures thereof.

Suitable solvents for the lipophilic agents include any non-polar or slightly polar solvent, such as t-butanol, ethanol, methanol, chloroform, or acetone that can be evaporated without leaving a pharmaceutically unacceptable residue. Drying can be by any suitable means such as by lyophilization, and preferably is performed in the presence of a cryoprotectant such as a non-reducing sugar. Preferred non-reducing sugars include glucose, maltose, lactose, sucrose and trehalose. Hydrophilic ingredients can be dissolved in polar solvents, including water.

Liposomes can be formed by mixing the dried lipophilic ingredients with the hydrophilic mixture. Mixing the polar solution with the dry lipid film can be by any means that strongly homogenizes the mixture. The homogenization can be effected by vortexing, magnetic stirring and/or sonicating.

Active agents also can be included in the liposomes containing PEGylated cardiolipin. Examples of active agents that can be included within the liposomes, in accordance with the inventive method, include one or more genetic vectors, antisense molecules, proteins, peptides, bioactive lipids or drugs. For example, the active agent can include one or more drugs (such as one or more anticancer drugs or other anticancer agents). An oligonucleotide (e.g., one or more oligonucleotides) also is a preferred active agent for inclusion in the liposomes, in accordance with the inventive method. Such oligonucleotides can be modified in manners known to those of ordinary skill in the art (e.g., phosphorothioated). Oligonucleotides for inclusion in the liposomal formulations include, for example, single stranded oligomers (e.g., antisense oligonucleotides) or double stranded oligonucleotides (e.g., siRNA).

Where such active agents are included, the method can permit such active agents to become entrapped within the liposomes or completed with the PEGylated cardiolipin present in the liposomes. Where such active agents are included in the liposomes, they can be dissolved or dispersed in a suitable solvent and added to the liposome mixture prior to mixing. Typically hydrophilic active agents will be added directly to the polar solvent and hydrophobic active agents will be added to the nonpolar solvent used to dissolve the other ingredients, but this is not required. The active agent could be dissolved in a third solvent or solvent mix and added to the mixture of polar solvent with the lipid film prior to homogenizing the mixture.

However prepared, the invention provides liposomes comprising PEGylated cardiolipin (such as PEGylated cardiolipin prepared in accordance with the methods disclosed herein or otherwise). In a preferred embodiment, the liposomal composition is present in a lyophilized form and can include one or more cryoprotectants. However, the composition also can include the liposomes in dispersion or other solvent system, as desired.

Generally, liposomes can have a net neutral, negative or positive charge. For example, positive liposomes can be formed from a solution containing phosphatidylcholine, cholesterol, cardiolipin and enough stearylamine to overcome the net negative charge of cardiolipin or cationic variants of cardiolipin can be used. Negative liposomes can be formed from solutions containing phosphatidylcholine, cholesterol, and/or cardiolipin, for example.

The liposomes of the present invention can be multi or unilamellar vesicles depending on the particular composition and procedure used to make them. Liposomes can be prepared to have substantially homogeneous sizes in a selected size range. Thus, for example, the liposomes of the present invention can have a diameter of about 1 micron or less, such as about 500 nm or less or even about 200 nm or less (e.g., 100 nm or less). One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane.

In addition to the PEGylated cardiolipin, liposomes according to the present invention can include stabilizers, absorption enhancers, antioxidants, lipophilic agents, biodegradable polymers, and medicinally active agents among other ingredients. Suitable antioxidants include compounds such as ascorbic acid, tocopherol, and deteroxime mesylate. Suitable absorption enhancers include Na-salicylate-chenodeoxy cholate, Na-deoxycholate, polyoxyethylene 9-lauryl ether, chenodeoxy cholate-deoxycholate and polyoxyethylene 9-lauryl ether, monoolein, Na-tauro-24,25-dihydrofusidate, Na-taurodeoxycholate, Na-glycochenodeoxycholate, oleic acid, linoleic acid, linolenic acid. Polymeric absorption enhancers can also be included such as polyoxyethylene ethers, polyoxyethylene sorbitan esters, polyoxyethylene 10-lauryl ether, polyoxyethylene 16-lauryl ether, azone (1-dodecylazacycloheptane-2-one). Preferably, the liposomes include a phophatidylcholine, a sterol, and a tocopherol, as lipophilic agents. Other lipophilic agents include phospholipids, phosphatidylglycerols, and sterols, such as those described above.

In preferred embodiments, the liposomes also include targeting agents, such as ligands that bind to a specific substrate. Suitable targeting agents include proteins (such as antibodies, antibody fragments, peptides, peptide hormones, receptor ligands and mixtures thereof) or carbohydrates. The inclusion of such agents can facilitate targeting the liposome to a predetermined tissue or cell type, for example, if the targeting agent is a ligand for a specific cellular receptor.

Suitable active agents that can be present in the inventive liposomal formulation include one or more genetic vectors, antisense molecules, proteins, peptides, bioactive lipids or drugs, such as are described above. In general, liposomes can be used to administer active agents that are stable in the presence of surfactants. Hydrophilic active agents are suitable and can be included in the interior of the liposomes such that the liposome bilayer creates a diffusion barrier preventing it from randomly diffusing throughout the body. Hydrophobic active agents are thought to be particularly well suited for use in the present method because they not only benefit by exhibiting reduced toxicity but they tend to be well solubilized in the lipid bilayer of liposomes.

Preferred active agents which are compatible with the present invention include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents can be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents can be analgesics, anesthetics, anti-arrythmic agents, antibiotics, antiallergic agents, antifungal agents, anticancer agents (e.g., mitoxantrone, taxanes, paclitaxel, camptothecin, and camptothecin derivatives (e.g., SN-38), gemcitabine, anthacyclines, antisense oligonucleotides, antibodies, cytoxines, immunotoxins, etc.), antihypertensive agents (e.g., dihydropyridines, antidepressants, cox-2 inhibitors), anticoagulants, antidepressants, antidiabetic agents, anti-epilepsy agents, anti-inflammatory corticosteroids, agents for treating Alzheimers or Parkinson's disease, antiulcer agents, anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents, immunosuppressive agents, anti-gout agents, anti-malarials, anti-migraine agents, antimuscarinic agents, anti-inflammatory agents, such as agents for treating rheumatology, arthritis, psoriasis, inflammatory bowel disease, Crohn's disease, or agents for treating demyelinating diseases including multiple sclerosis, ophthalmic agents; vaccines (e.g., against influenza virus, pneumonia, hepatitis A, hepatitis B, hepatitis C, cholera toxin B-subunit, typhoid, plasmodium falciparum, diptheria, tetanus, herpes simplex virus, tuberculosis, HIV, bordetela pertusis, measles, mumps, rubella, bacterial toxoids, vaccinea virus, adenovirus, SARS virus, canary virus, bacillus calmette Guerin, klebsiella pneumonia vaccine, etc.), histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., androgens, estrogens, etc.), stimulants, sympathomimetics, vasodilators and xanthins and synthetic analogs of these species. The therapeutic agents can be nephrotoxic, such as cyclosporins and amphotericin B, or cardiotoxic, such as amphotericin B and paclitaxel. Exemplary anticancer agents include melphalan, chlormethine, extramustinephosphate, uramustine, ifosfamide, mannomustine, trifosfamide, streptozotocin, mitobronitol, mitoxantrone, methotrexate, fluorouracil, cytarabine, tegafur, idoxide, taxol, paclitaxel, daunomycin, daunorubicin, bleomycin, amphotericin, carboplatin, cisplatin, paclitaxel, taxotere, BCNU, vincristine, camptothecin, SN-38, doxorubicin, etopside, cytokines, ribozymes, interferons, oligonucleotides, siRNAs, RNAis and functional derivatives of the foregoing. Additional examples of drugs which can be delivered according to the method include prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isofluorophate, acetazolamide, methazolamide, bendroflumethiazide, chloropromaide, tolazamide, chlomadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, heparin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, somatotropins (e.g., bovine somatotropin, porcine somatotropin, etc.), oxytocin, vasopressin, GRF, somatostatin, lypressin, pancreozymin, luteinizing hormone, LHRH, LHRH agonists and antagonists, leuprolide, interferons (e.g., α-, β-, or γ-interferon, interferon α-2a, interferon α-2b, and consensus interferon, etc.), interleukins, growth hormones (e.g., human growth hormone and its derivatives such as methione-human growth hormone and des-phenylalanine human growth hormone, bovine growth hormone, porcine growth hormone, insulin-like growth hormone, etc.), fertility inhibitors such as the prostaglandins, fertility promoters, growth factors such as insulin-like growth factor, coagulation factors, pancreas hormone releasing factor, analogs and derivatives of these compounds, and pharmaceutically acceptable salts of these compounds, or their analogs or derivatives. The therapeutic agent can be a mixture of agents that can be beneficially co-administered in the liposome formulation.

Chemotherapeutic agents are well suited for use in the method. Liposome formulations containing chemotherapeutic agents can be injected directly into the tumor tissue for delivery of the chemotherapeutic agent directly to cancer cells. In some cases, particularly after resection of a tumor, the liposome formulation can be implanted directly into the resulting cavity or can be applied to the remaining tissue as a coating. In cases in which the liposome formulation is administered after surgery, it is possible to utilize liposomes having larger diameters of about 1 micron since they do not have to pass through the vasculature.

In addition to the foregoing ingredients, the liposomal formulations of the present invention also can include one or more pharmaceutically-acceptable agents, such as buffers, excipients, and other agents known for use in pharmaceutics. Thus, the invention provides for the administration of pharmaceutical preparations which, in addition to liposome formulations of active agents, include non-toxic, inert pharmaceutically suitable excipients. Pharmaceutically suitable excipients include solid, semi-solid or liquid diluents, fillers and formulation auxiliaries of all kinds. The invention also includes pharmaceutical preparations in dosage units. This means that the preparations are in the form of individual parts, for example vials, syringes, capsules, pills, suppositories, or ampoules, of which the content of the liposome formulation of active agent corresponds to a fraction or a multiple of an individual dose. The dosage units can contain, for example, 1, 2, 3, or 4 individual doses, or ½, ⅓, or ¼ of an individual dose. An individual dose preferably contains the amount of active agent which is given in one administration and which usually corresponds to a whole, a half, a third, or a quarter of a daily dose. It is within the ordinary skill of the art to select a dosage of active agent within the inventive composition suitable for a given therapeutic application.

Tablets, dragees, capsules, pills, granules, suppositories, solutions, suspensions and emulsions, pastes, ointments, gels, creams, lotions, powders and sprays can be suitable pharmaceutical preparations. Suppositories can contain, in addition to the liposomal active agent, suitable water-soluble or water-insoluble excipients. Suitable excipients are those in which the inventive liposomal active agent is sufficiently stable to allow for therapeutic use, for example polyethylene glycols, certain fats, and esters or mixtures of these substances. Ointments, pastes, creams and gels can also contain suitable excipients in which the liposomal active agent is stable.

The invention also is directed to methods of delivering active agents to cells. The method can be carried out by preparing liposomes that include active agents and PEGylated cardiolipin analogues (e.g., as synthesized by the above disclosed methods or otherwise). The liposomes are then delivered to a cell. For in vitro use, this can be carried out by adding the liposomes to the cell culture medium for example.

The method can be used to deliver the active agent to cells in vivo as well. For example, the composition can be delivered orally, by injection (e.g., intravenously, subcutaneously, intramuscularly, parenterally, intraperitoneally, by direct injection into tumors or sites in need of treatment, etc.) by inhalation, by mucosal delivery, locally, and/or rectally or by such methods as are known or developed. Formulations containing PEGylated cardiolipin can also be administered topically, e.g., as a cream, skin ointment, dry skin softener, moisturizer, etc.

For in vivo use, the invention provides the use of a composition as herein described containing one or more active agents for preparing a medicament for the treatment of a disease. In other words, the invention provides a method of using a composition as herein described, containing one or more active agents, for treating a disease. Typically, the disease is present in a human or animal patient. In a preferred embodiment, the disease is cancer, in which instance, the inventive composition comprises one or more anticancer agents as active agents. For example, in accordance with the invention, the compositions as described herein can be employed alone or adjunctively with other treatments (e.g., chemotherapy or radiotherapy) to treat cancers such as those of the head, neck, brain, blood, breast, lung, pancreas, bone, spleen, bladder, prostate, testes, colon, kidney, ovary and skin. The compositions of the present invention, comprising one or more anticancer agents, are especially preferred for treating leukemias, such as acute leukemia (e.g., acute lymphocytic leukemia or acute myelocytic leukemia). Kaposi's sarcoma also can be treated using the compositions and methods of the present invention.

It should be noted that, where the inventive compositions are employed to treat diseases (e.g., cancer) in human or animal patients, they need not result in a complete cure or remission of the disease to be shown to be successfully employed. Thus, for example, the compositions can be successfully employed if, by using the inventive composition, the progress of the disease is slowed or retarded in the patient. Alternatively, the inventive composition is deemed to have been used successfully in the treatment of the disease if, for adjunctive uses, the inventive composition renders the disease more amenable to other treatment or demonstrates an additive, but not necessarily synergistic, therapeutic potential as compared to monotherapy using the other treatment regimen. However, in some embodiments, the use of the composition in accordance with the present invention can lead to remission of a cancer or other disease.

The following examples further illustrate the invention but, of course, should not be constructed as in any way limiting its scope.

### EXAMPLE 1

### 1A. 1,3-bis[(1,2-dimyristoyl-sn-glycero-3)-phosphoryl]-2-succinyl glycerol ester

To a solution of 1,3-bis(1,2-dimyristoyl-sn-glycero-3)-phosphorylglycerol dibenzylester (3.5 g, 2.46 mmol) in 1,2-dichloroethane (30 mL) at room temperature were added succinic anhydride (615 mg, 6.15 mmol) and triethylamine (1.67 mL, 12.0 mmol) sequentially and stirred for 6 h. The reaction mixture was diluted with dichloromethane and neutralized with 1N HCl until the aqueous layer was just acidic (pH 6-7). The organic layer was separated, dried (Na₂SO₄) and concentrated. The residue was purified on SiO₂ (20% acetone in dichloromethane) to give 3.01 g (80%) of the product as colorless syrup. TLC (SiO2) hexane/acetone (3:2) Rf˜0.37. ¹H NMR δ (CDCl₃), 500 MHz) 0.88 (t, J=7.0 Hz, 12H), 1.22-1.34 (m, 80H), 1.54-1.63 (m, 8H), 2.24-2.31 (m, 8H), 2.60-2.68 (m, 4H), 4.05-4.30 (m, 13H), 5.02-5.11 (m, 4H), 5.14-5.21 (m, 2H), 7.31-7.39 (m, 10H).

### 1B. 1,3-bis[(1,2-dimyristoyl-sn-glycero-3)-phosphoryl]-2-succinimidyl succinate glycerol dibenzyl ester (cardiolipin dibenzyl ester succinimidyl succinate)

To a solution of 1,3-bis[(1,2-dimyristoyl-sn-glycero-3)-phosphoryl]-2-succinyl glycerol ester (1.5 g, 0.98 mmol) in dry THF (20 mL), N-hydroxysuccinimide (NHS; 198 mg, 1.72 mmol) and DCC (460 mg, 2.2 mmol) were added at room temperature and stirred for 12 h. The volatiles were removed in vacuo and the residue was purified by SiO₂ column chromatography (5% methanol in dichloromethane) to give 1.35 g (84%) of the product as colorless syrup. TLC (SiO₂) hexane/acetone (3:2) Rf˜0.52.

### 1C. 1,3-bis[(1,2-dimyristoyl-sn-glycero-3)-phosphoryl]-2-mPEG succinamido glycerol dibenzyl ester

To a solution of 1,3-bis[(1,2-dimyristoyl-sn-glycero-3)-phosphoryl]-2-succinimidyl succinate glycerol dibenzyl ester (1.35 g, 0.83 mmol) and DMAP (92 mg, 0.74 mmol) in dry THF (10 mL), PEG-amine 2000 (1.25 g, 0.62 mmol) or (PEG-amine 5000) was added at room temperature and stirred for 24 h. The volatiles were removed in vacuo, the residue was dissolved in dichloromethane (200 mL), washed with water (100 mL), dried (Na₂SO₄) and concentrated. The residue was purified on SiO₂ (6% methanol in dichloromethane) to give 1.75 g (60%) of the CL-conjugate as white solid. TLC (SiO₂) dichloromethane/methanol (9:1) R_{f}˜0.49. ¹H NMR δ (CDCl₃, 500 MHz) 0.88 (t, J=7.0 Hz, 12H), 1.22-1.34 (m, 80H), 1.54-1.63 (m, 8H), 2.24-2.31 (m, 8H), 2.40-2.48 (m, 2H), 2.57-2.68 (m, 2H), 3.40-3.44 (m, 2H), 3.49-3.55 (m, 4H), 3.57-3.68 (m, 175H), 4.05-4.30 (m, 13H), 5.02-5.11 (m, 4H), 5.14-5.21 (m, 2H), 6.23-6.32 (m, 1H), 7.31-7.39 (m, 10H). Mass spectrum (MALDI) average m/z calculated for C₁₇₂H₃₃₇NO₆₉P₂: 3583 Found: 3583.

### 1D. 1,3-bis[(1,2-dimyristoyl-sn-glycero-3)-phosphoryl]-2-mPEG succinamido glycerol diammonium salt (MPEG 2000-succ-CL)

A solution of 1,3-bis[(1,2-dimyristoyl-sn-glycero-3)-phosphoryl]-2-mPEG succinamido glycerol dibenzyl ester (1.02 g, 0.29 mmol) in tetrahydrofuran (25 mL) was hydrogenated at 50 psi over 10% Pd/C (200 mg) for 4 h. The catalyst was filtered off over celite bed, treated with 2 mL of 30% ammonia solution and concentrated, the residue was purified on SiO₂ (12% methanol in dichloromethane and 1% triethylamine) to give 790 mg (81%) of the PEG-CL as white solid. TLC (SiO₂) CHCl₃/MeOH/NH₄OH (7.0:2.5:0.5) R_{f}˜0.48. ¹H NMR δ (CDCl₃, 500 MHz) 0.88 (t, J=7.0 Hz, 12H), 1.22-1.34 (m, 80H), 1.54-1.63 (m, 8H), 2.24-2.31 (m, 8H), 2.42-2.58 (m, 2H), 2.64-2.72 (m, 2H), 3.40-3.68 (m, 181H), 3.76-3.93 (m, 1H), 3.87-4.24 (m, 10H), 4.29-4.39 (m, 2H), 4.60, 5.06 (2t, 1H), 5.14-5.23 (m, 2H), 7.37 (bs, 8H). (mPEG 2000-succ-CL) Mass spectrum (MALDI) average m/z calculated for C₁₇₀H₃₃₉N₃O₆₉P₂: 3589 Found: 3579. (mPEG 5000-succ-CL) Mass spectrum (MALDI) average m/z calculated for C₃₁₈H₆₃₅N₃O₁₄₃P₂: 6845 Found: 6838.

### EXAMPLE 2

### 2A. 1,3-bis[1,2-dimyristoyl-sn-glycero-3-phosphoryl]-2-(ter-butoxycarbonyl)glycinyl glycerol dimethyl ester

A solution of 1,2-dimyristoyl-sn-glycerol (6.52 g, 12.74 mmol), methyl N,N-tetraisopropyl phosphoramidite (3.34 g, 12.74 mmol) and 1H-tetrazole (28.3 mL of 0.45 M sol in acetonitrile, 12.74 mmol) in CH₂Cl₂ (25 mL) was stirred at room temperature under argon for 3 h. A solution of 2-(ter-butoxycarbonyl)glycinyl-1,3-propanediol (1.41 g, 5.66 mmol) in CH₂Cl₂ (10 mL) was added followed by 1H-tetrazole (28.3 mL of 0.45 M Sol in acetonitrile, 12.74 mmol) and stirred for 3 h. The reaction mixture was cooled to −40° C. and tert-butylhydroperoxide (TBHP, 3.47 mL of 5.0-6.0M sol in decane, 19.11 mmol) was added. After stirring at −40° C. for 30 minutes, the reaction mixture was warmed to room temperature, diluted with CH₂Cl₂ (250 mL), washed (saturated aqueous Na₂SO₃ (2×50 mL), saturated aqueous NaHCO₃ (2×50 mL), brine (2×50 mL)), dried (Na₂SO₄), and concentrated. The residue was purified on SiO₂ (2:3 EtOAc:hexane) to give 1.87 g (21%) of protected cardiolipin as colorless syrup. TLC (SiO₂) hexane/EtOAc (1:1) R_{f}˜0.42. ¹H NMR δ (CDCl₃, 500 MHz) 0.88 (t, J=7.0 Hz, 12H), 1.22-1.34 (m, 80H), 1.45 (s, 9H), 1.54-1.63 (m, 8H), 2.28-2.36 (m, 8H), 3.76-3.83 (m, 6H), 3.92-3.97 (m, 2H), 4.07-4.30 (m, 10H), 4.31-4.36 (m, 2H), 4.41-4.45 (m, 1H), 4.71-4.75 (m, 1H), 5.18-5.26 (m, 2H).

### 2B. 1,3-bis[(1,2-dimyristoyl-sn-glycero-3)-phosphoryl]-2-mPEG succinamido glycinyl glycerol dimethyl ester

To a solution of 1,3-bis[1,2-dimyristoyl-sn-glycero-3-phosphoryl]-2-(ter-butoxycarbonyl)glycinyl glycerol dimethyl ester (1.3 g, 0.83 mmol) in CH₂Cl₂ (9 mL) at 0° C., trifluoroacetic acid was added and stirred for 2 h and then gradually allowed to attain room temperature. The reaction mixture was diluted with dichloromethane (30 mL) and neutralized with aqueous NaHCO₃ solution. The organic layer was separated, dried (Na₂SO₄) and concentrated and the resulting amine (930 mg) was subjected to the next reaction without any purification. TLC (SiO₂) hexane/acetone (1:1) R_{f}˜0.12.

To a solution of above amine (930 mg, 0.7 mmol) and DMAP (85 mg, 0.7 mmol) in dry THF (10 mL), PEG-SG 2000 (1.1 g, 0.56 mmol) was added at room temperature and stirred for 24 h. The volatiles were removed in vacuo, the residue was dissolved in dichloromethane (200 mL), washed with water (100 mL), dried (Na₂SO₄) and concentrated. The residue was purified on SiO₂ (10% methanol in dichloromethane) to give 830 mg (35%) of the CL-conjugate as white solid. TLC (SiO₂) dichloromethane/methanol (9:1) Rf˜0.39. ¹H NMR δ (CDCl₃, 500 MHz) 0.88 (t, J=7.0 Hz, 12H), 1.22-1.34 (m, 80H), 1.54-1.63 (m, 8H), 1.92-1.99 (m, 2H), 2.28-2.36 (m, 8H), 2.38-2.44 (m, 4H), 3.38-3.83 (m, 188H), 4.01-4.42 (m, 13H), 4.71-4.75 (m, 1H), 5.20-5.29 (m, 2H).

### 2C. 1,3-bis[(1,2-dimyristoyl-sn-glycero-3)-phosphoryl]-2-mPEG succinamido glycinyl glycerol diammonium salt (mPEG 2000-Gly-CL)

A solution of 1,3-bis[(1,2-dimyristoyl-sn-glycero-3)-phosphoryl]-2-mPEG succinamido glycinyl glycerol dimethyl ester (850 mg, 0.25 mmol) in 2-butanone (10 mL) and sodium iodide (114 mg, 0.766 mmol) was refluxed at 90° C. for 3 h. The volatiles were evaporated and the residue was purified on SiO₂ (15% methanol in CH₂Cl₂ containing 1% of ammonia) to give 75 mg (15%) of the product as colorless semisolid. TLC (SiO₂) CHCl₃/MeOH/NH₄OH (7.0:2.5:0.5) R_{f}˜0.54 TLC (SiO₂) ¹H NMR δ (CDCl₃, 500 MHz) 0.88 (t, J=7.0 Hz, 12H), 1.22-1.34 (m, 80H), 1.54-1.63 (m, 8H), 1.92-1.99 (m, 2H), 2.28-2.36 (m, 8H), 2.38-2.44 (m, 4H), 3.38-3.83 (m, 182H), 4.01-4.42 (m, 13H), 4.57-4.68 (m, 1H), 5.20-5.29 (m, 2H), 7.29 (bs, 8H).

### EXAMPLE 3

### 3A. 1,3-bis[(1,2-dimyristoyl-sn-glycero-3)-phosphoryl]-2-(ter-butoxycarbonyl)-glycinyl glycerol dibenzyl ester

To a solution of 1,2-Dimyristoyl-sn-glycerol (10.0 g, 19.53 mmol) and tetrazole (52 mL of 0.45 M sol in acetonitrile, 23.43 mmol) in 150 mL anhydrous CH₂Cl₂, dibenzyl diisopropyl phosphoramidite (7.06 g, 21.48 mmol) was added and stirred at room temperature for 2 h. The contents were diluted with 100 mL of CH₂Cl₂ and then washed with 5% aqueous NaHCO₃ (2×50 mL), brine (2×50 mL), dried over Na₂SO₄, concentrated in vacuo and the oily residue (14.7 g) was dried in a desiccator for 8 h and used as such in the next reaction.

A solution of above phosphite, 2-(ter-butoxycarbonyl)glycinyl-1,3-propanediol (1.7 g, 6.83 mmol), pyridine (15.7 mL, 195.3 mmol) and Et₃N (13.58 mL, 97.65 mmol) in CH₂Cl₂ (180 mL) was cooled to −40° C. and pyridinium tribromide (9.4 g, 29.79 mmol) was added at a time. The mixture was stirred at the same temperature for 1 h and gradually allowed to attain room temperature over a period of 2 h and treated with water (30 mL). The contents were diluted with EtOAc (250 mL) and the organic layer was washed successively with aqueous 5% NaHCO₃ (2×50 mL), water (50 mL) and brine (50 mL), dried (Na₂SO₄) and concentrated. The residue was purified on SiO₂ (2% methanol in dichloromethane) to give 1.85 g (18%) of the product as colorless syrup. TLC (SiO₂) hexane/EtOAc (1:21) Rf˜0.28. ¹H NMR δ (CDCl₃, 500 MHz) 0.88 (t, J=7.0 Hz, 12H), 1.22-1.34 (m, 80H), 1.45 (s, 9H), 1.54-1.63 (m, 8H), 2.28-2.36 (m, 8H), 3.92-3.97 (m, 2H), 4.02-4.33 (m, 13H), 4.57-4.62 (m, 1H), 5.02-5.12 (m, 4H), 5.18-5.26 (m, 2H), 7.31-7.42 (m, 10H).

### 3B. 1,3-bis[(1,2-dimyristoyl-sn-glycero-3)-phosphoryl]-2-mPEG succinamido glycinyl glycerol dibenzyl ester

To a solution of 1,3-bis[(1,2-dimyristoyl-sn-glycero-3)-phosphoryl]-2-(ter-butoxycarbonyl)-glycinyl glycerol dibenzyl ester (1.3 g, 0.83 mmol) in CH₂Cl₂ (9 mL) at 0° C., trifluoroacetic acid was added and stirred for 2 h and then gradually allowed to attain room temperature. The reaction mixture was diluted with dichloromethane (30 mL) and neutralized with aq NaHCO₃ solution. The organic layer was separated, dried (Na₂SO₄) concentrated and the resulting amine (930 mg) was subjected to next reaction without any purification. TLC (SiO₂) hexane/acetone (1:1) R_{f}˜0.12.

To a solution of above amine (930 mg, 0.7 mmol) and DMAP (85 mg, 0.7 mmol) in dry THF (10 mL), PEG-SG 2000 (1.1 g, 0.56 mmol) was added at room temperature and stirred for 24 h. The volatiles were removed in vacuo, the residue was dissolved in dichloromethane (200 mL), washed with water (100 mL), dried (Na₂SO₄) and concentrated. The residue was purified on SiO₂ (10% methanol in dichloromethane) to give 830 mg (35%) of the CL-conjugate as white solid.

### 3C. 1,3-bis[(1,2-dimyristoyl-sn-glycero-3)-phosphoryl]-2-mPEG succinamido glycinyl glycerol diammonium salt (mPEG 2000-Gly-CL)

A solution of 1,3-bis[(1,2-dimyristoyl-sn-glycero-3)-phosphoryl]-2-mPEG succinamido glycinyl glycerol dibenzyl ester (640 mg, 0.18 mmol) in tetrahydrofuran (20 mL) was hydrogenated at 50 psi over 10% Pd/C (100 mg) for 4 h. The catalyst was filtered off over celite bed, treated with 2 mL of 30% ammonia solution and concentrated, the residue was purified on SiO₂ (15% methanol in dichloromethane and 1% triethylamine) to give 55 mg (10%) of the PEG-CL as white solid. TLC (SiO₂) CHCl₃/MeOH/NH₄OH (7.0:2.5:0.5) R_{f}˜0.48. ¹H NMR δ (CDCl₃, 500 MHz) 0.88 (t, J=7.0 Hz, 12H), 1.22-1.34 (m, 80H), 1.54-1.63 (m, 8H), 1.92-1.99 (m, 2H), 2.28-2.36 (m, 8H), 2.38-2.44 (m, 4H), 3.38-3.83 (m, 182H), 4.01-4.42 (m, 13H), 4.57-4.68 (m, 1H), 5.20-5.29 (m, 2H), 7.29 (bs, 8H).

### EXAMPLE 4

### A. 1,3-bis[(1,2-dimyristoyl-sn-glycero-3)-phosphoryl]-2-mPEG propionamido glycinyl glycerol dimethyl ester (mPEG 2000-Gly-CL)

To a solution of amine (600 mg, 0.45 mmol) and DMAP (10 mg, 0.09 mmol) in anhydrous THF (10 mL) was added mPEG-SPA (2000) (370 mg, 0.18 mmol) and stirred at room temperature for 48 hours. The solvents were removed and the residue was dissolved in CH₂Cl₂ (75 mL) and washed with water. The organic layer was dried over sodium sulfate and concentrated. The residue was purified by silica gel column chromatography using 10% methanol in CH₂Cl₂ as eluent to yield the product as white solid. Yield was 450 mg. ¹H NMR δ (CDCl₃, 500 MHz) 0.88 (t, J=7.0 Hz, 12H), 1.22-1.34 (m, 80H), 1.54-1.63 (m, 8H), 2.28-2.36 (m, 8H), 2.38-2.44 (m, 4H), 3.38-3.83 (m, 188H), 4.01-4.42 (m, 13H), 4.71-4.75 (m, 1H), 5.20-5.29 (m, 2H).

### 4B. 1,3-bis[(1,2-dimyristoyl-sn-glycero-3)-phosphoryl]-2-mPEG propionamido glycinyl glycerol diammonium salt (mPEG 2000-Gly-CL)

A solution of 1,3-bis[(1,2-dimyristoyl-sn-glycero-3)-phosphoryl]-2-mPEG propionamido glycinyl glycerol dimethyl ester (450 mg, 0.134 mmol) in 2-butanone (15 mL) was heated for reflux with NaI (60 mg 0.40 mmol) for 3 hours at 90° C. The volatiles were removed on rotavapor and the residue was purified by silica gel chromatography using CH₂Cl₂/MeOH/NH₄OH (89:10:1) to yield 250 mg of PEG-Cl as white solid. ¹H NMR δ (CDCl₃, 500 MHz) 0.88 (t, J=7.0 Hz, 12H), 1.22-1.34 (m, 80H), 1.54-1.63 (m, 8H), 2.28-2.36 (m, 8H), 2.38-2.44 (m, 4H), 3.38-3.83 (m, 182H), 4.01-4.42 (m, 13H), 4.57-4.68 (m, 1H), 5.20-5.29 (m, 2H), 7.29 (bs, 8H)

All references, including publications, patent applications, and patents, cited herein, including those in the following list and otherwise cited in this specification, are hereby incorporated by reference to the same extent as if each reference is were individually and specifically indicated to be incorporated by reference and were set forth in its entirety herein.
Martin, F. J.; Woodle, M. C.; Redemann, C.; Yau-Young, A.; US Patent App. 20030113369.
Harrington, K. J., Rowlinson-Busza, G., Syrigos, K. N., Uster, P. S., Vile, R. G., Stewart, S. W. Clinical Cancer Research, 6, 2528-2537 (2000). Fisher, D., U.S. Pat. No. 6,132,763.
Harrington, K. J., Mohammadtagi, S., Uster, P. S., Glass, D., Peters, M., Vile, R. G.,
Stewart, S. W. Clinical Cancer Research, 7, 243-254 (2001).
Huang, S. K., Lee, K-D., Hong, K., friend, D. S., Papahadjopoulos, D. Cancer Res. 52, 5135-5143 (1992).
Huang, S. K., Martin, F. J., Jay, G., Vogel, J., Papahadjopoulos, Friend, D S, Am. J. Pathol., 143, 10-14 (1993).
Harrington, K. J., Lewanski, C. R., Stewart, J. S. Clin. Oncol. (R. Coll. Radiol.), 12, 2-15, 2000.
Working, P. K., Newman, M. S., Sullivan, T., Yarrington, J. J. Pharmacol. Exp. Ther. 289, 1128-1133, (1999).
Gabizon, A., Goren, D., Cohen, R., Barenholz, Y., J. Controlled Release, 53, 275-279 (1998).
Woodle M. C., Lasic, D. D., Biochim. Biophys. Acta, 1113, 171 (1992).
Lasic, D. D., Needham, D., Chem. Rev., 95, 2601 (1995).
Ahmad, M. U., Ukkalam, M. U., Ahmad, I. US/PCT03/27806.
Ahmad, M. U., Lin, Z., Ali, S. M., Ahmad, I. US/PCT03/16412
Krishna, U. M., Ahmad, M. U., Ahmad, I. Tetrahedron Lett. 45, 2077-2079 (2004).

The use the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having”, “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specifications should be construed as indicating any non-claimed element as essential to the practice of the invention.

Preferred embodiments of this invention are described herein, including the best mode known to the inventors for carrying out the invention. Variations of those preferred embodiments can become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A PEGylated cardiolipin molecule of structure I. 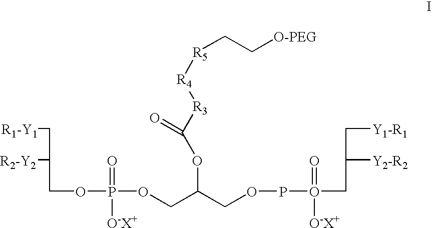
wherein Y₁ and Y₂ are the same or different and are —O—C(O)—, —O—, —S—, —NH—C(O)— or the like;
R₁ and R₂ are the same or different and are H, saturated alkyl group and/or unsaturated alkyl group;
R₃, R₄, R₅ are the same or different and are O, CO, NR wherein R is H, an alkyl group ranging from C₁-C₁₀, (CH₂)ₙ where n=0-10 or substituted (CH₂)ₙ where n=0-10.
X is hydrogen, ammonium, sodium, potassium, calcium, barium ion or any non-toxic cation; and
the PEG group (polyethylene glycol) is a long chain, linear or branched synthetic polymer.

2. A PEGylated cardiolipin molecule of structure II. 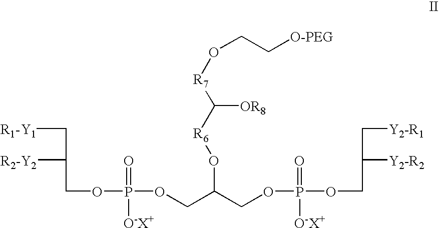
wherein Y₁ and Y₂ are the same or different and are —O—C(O)—, —O—, —S—, —NH—C(O)— or the like,
R₁ and R₂ are the same or different and are H, saturated alkyl group and/or unsaturated alkyl group,
R₆ and R₇ are the same or different and are (CH₂)ₙ where n=1-10 or substituted (CH₂)ₙ where n=1-10,
R₈ is H, alkyl, substituted alkyl, cycloalkyl, substituted cycloalkyl, alkyloxy or polyalkyloxy group,
X is hydrogen, ammonium, sodium, potassium, calcium, barium ion or any non-toxic cation, and
the PEG (polyethylene glycol) group is a long chain, linear synthetic polymer.

3. (canceled)

4. (canceled)

5. The PEGylated cardiolipin molecule as in claim 1 or 2 where the PEG group is composed of ethylene oxide units, HO(CH₂CH₂O)ₙCH₂CH₂OCH₃, in which n=1-500.

6. The PEGylated cardiolipin molecule of claim 5 wherein the PEG group is substituted with a methyl group at the terminal position.

7. The PEGylated cardiolipin molecule of any of claims 1-2 wherein at least one of R₁ or R₂ is a saturated or unsaturated alkyl group having between 1 and 34 carbon atoms.

8. (canceled)

9. The PEGylated cardiolipin molecule of any of claims 1-2 wherein at least one of R₁ or R₂ is a saturated or unsaturated alkyl group having between 12 and 24 carbon atoms.

10. A method of preparing PEGylated cardiolipin and analogues thereof, comprising reacting any functional group of cardiolipin or functional group of any linker with a PEG-reagent.

11. (canceled)

12. (canceled)

13. (canceled)

14. A method for preparing PEGylated cardiolipin and analogues thereof, comprising linking a PEG to the central glycerol unit of a cardiolipin molecule or analogues thereof.

15. The method of claim 14, further comprising:
a. attaching a linker to said cardiolipin molecule and analogues thereof by reacting the central hydroxyl unit of formula 1 with a cyclic anhydride in an inert solvent in the presence of a base, wherein R₁ and R₂ are the same or different and are H, a saturated alkyl group and/or an unsaturated alkyl group and A is a protecting group, 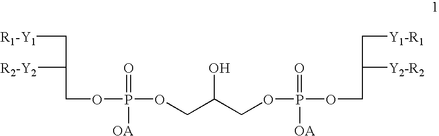
b. reacting formula 1 containing a linker, with PEG-reagent containing a reactive functional group and
c. removing the protecting groups A.

16. (canceled)

17. (canceled)

18. (canceled)

19. (canceled)

20. (canceled)

21. (canceled)

22. (canceled)

23. The method of claim 14, further comprising:
a. reacting central hydroxyl group of cardiolipin precursor of formula 1 and a reactive functional group of a PEG reagent, wherein R₁ and R₂ are the same or different and are H, a saturated alkyl group and/or an unsaturated alkyl group and R is a protecting group and

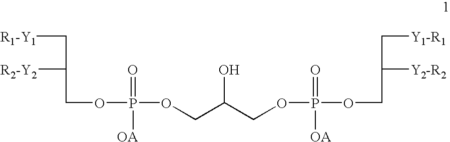

24. The method of claim 23, wherein the PEG reagent is selected from PEGylated p-nitrophenyl carbonate (PEG-NPC) of formula 14, 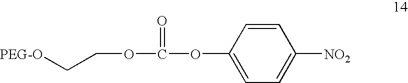
PEGylated isocynate (PEG-NC) of formula 15, 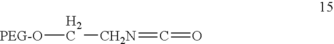
and PEGylated epoxide of formula 16.

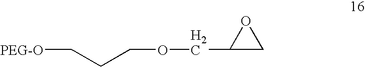

25. (canceled)

26. (canceled)

27. The method for preparing PEGylated cardiolipin and analogues thereof, comprising:
a. reacting a phosphoramidite derivative of 1,2-substituted glycerol of formula 7, and a 2-substituted glycerol of formula 8 in the presence of an agent, where the agent is either 1-H-tetrazole or 4,5-dicyanoimidazole or the like and wherein R₁ and R₂ are the same or different and are H, a saturated alkyl group and/or an unsaturated alkyl group, A is a protecting group, R₃, R₄, R₅ are the same or different and are —O—, —C(O)—, —NR— wherein R is H or an alkyl group ranging from C₁-C₁₀, (CH₂)ₙ where n=0-10 or substituted (CH₂)ₙ where n=0-10 and B is a protecting group
b. removing the protecting group B with an acid in an inert solvent,
c. reacting the free amino group with a reactive functional group of a PEG reagent in the presence of DMAP in an inert solvent and
d. removing the protecting group A.

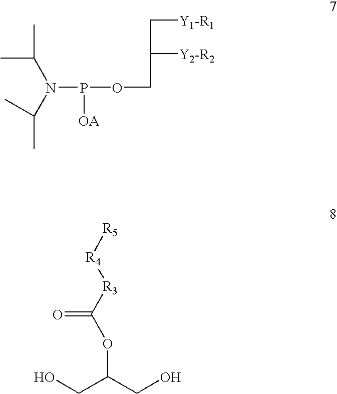

28. A method for preparing PEGylated cardiolipin and analogues thereof, comprising:
a. reacting a phosphoramidite derivative of 1,2-substituted glycerol of formula 10 and a 2-substituted glycerol of formula 8 in the presence of pyridinium perbromide in an inert solvent or the like and containing a suitable base, wherein R₁ and R₂ are the same or different and are H, a saturated alkyl group and/or an unsaturated alkyl group, A and B are protecting groups and R₃, R₄, R₅ are the same or different and are —O—, —C(O)—, —NR— wherein R is H or an alkyl group ranging from C₁-C₁₀, (CH₂)ₙ where n=0-10 or substituted (CH₂)ₙ where n=0-10.
b. Removing the protecting group B with an acid in an inert solvent,
c. Reacting the free amino group with a reactive functional group of a PEG reagent in the presence of DMAP in an inert solvent and
d. Removing the protecting group A.

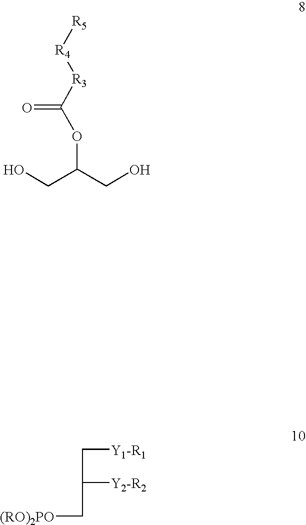

29. The method of claim 27 or 28, wherein the PEG reagent is selected from PEG-NHS, PEG-SG, and PEG-SPA.

30. (canceled)

31. (canceled)

32. (canceled)

33. (canceled)

34. A method for retaining one or more active agents in a liposome, comprising preparing a PEGylated cardiolipin by any of the methods of claims 10 or 14 and including said PEGylated cardiolipin and at least one active agent in a liposome.

35. The method of claim 34, wherein at least one active agent is entrapped within the liposome.

36. The method of claim 34, wherein at least one active agent is complexed with PEGylated cardiolipin.

37. The method of any of claim, 34 wherein the active agent includes an anticancer agent.

38. (canceled)

39. (canceled)

40. (canceled)

41. A composition comprising a PEGylated cardiolipin produced in accordance with the method of any of claims 10 or 14.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)
